(19) United States
(12) Patent Application Publication
(10) Pub. No.: US 2008/0050729 A1
(43) Pub. Date: Feb. 28, 2008

(54) Complex cancer cells luminescence detection method

(75) Inventors: Shiu-Ru Lin, Kaohsiung City (TW); Tian-Lu Cheng, Kaohsiung City (TW)

Correspondence Address: TROXELL LAW OFFICE PLLC, SUITE 1404, 5205 LEESBURG PIKE, FALLS CHURCH, VA, 22041, US

(73) Assignee: Kaohsiung Medical University, Kaohsiung City (TW)

(21) Appl. No.: 11/585,974

(22) Filed: Oct. 25, 2006

(30) Foreign Application Priority Data
Aug. 24, 2006 (TW) 095131098

(51) Int. Cl. C12Q 1/68

(52) U.S. Cl. 435/6

(57) ABSTRACT

A method for detecting cancer cells. A chemical luminescence reaction is used to obtain a result image. The result image is analyzed through a weighting process to a value of each gene. By doing do, a cancer can be precisely diagnosed.

### FIELD OF THE INVENTION

The present invention relates to a detection method; more particularly, relates to using a low-cost gene chip to precisely diagnose the cancer with an easy operated luminescence analysis and by weighting value for each gene according to its various role playing in the forming of the cancer.

### DESCRIPTION OF THE RELATED ARTS

An organism chip is a high-tech chip, which is fabricated with a substrate of silicon chip, glass, nylon membrane or polymer organic plastics based on molecular biology or analytical chemistry coordinated with technology of micro-system or any other precision machining technology. The organism chip has a small size, a fast reaction and a great amount of parallel analysis to biological data. Hence, the organism chip is widely used in drug development, medical analysis, disease screening, pathogeny identification, environmental analysis, food inspection, military detection, etc.

Among organism chips, gene chip is the most rapid developed, mature, and attention-getting. And there are two kinds of gene chips:
(1) Glass carrier chip: After a glass chip is processed through a specific procedure, nucleotide fragments are rapidly and densely dotted by a mechanical arm on the glass carrier in a matrix form to obtain a gene carrier chip. The glass carrier chip has some disadvantages preventing it from wide pervading in related fields:
   i) A high technology is required to process the glass carrier chip. And the cost of the chip is not afforded by a general laboratory.
   (ii) Dust and particles might seriously affects the dotting of the chip and the succeeding luminescence labeling; and so the result might have a noticeable deviation.
   (iii) A specimen (nucleotide) required for luminescence reaction and hybridization reaction of the glass carrier chip has to have a high quality. The reactions have complex steps and tough technology. The luminescence signal matter required, such as cy3 or cy5, has a cost two to five times to a general signal matter. And the preservation period of the required signal matter is short after using.
   (iv) And, the result obtained after the luminescence labeling requires a specific scanner for analysis, where the scanner is not affordable by a general laboratory.
(2) Nylon membrane chip: A nylon membrane is fixed with nucleotide fragments to obtain a gene chip. On comparing to the glass carrier chip, the nylon membrane chip is easily fabricated, has low technology required and uses a simple colorimetric method and a cheap labeling reagent. But the colorimetric method used is not automatic; and so a sensibility and a precision are in lack. And so, in turn, the nylon membrane chip is gradually rep laced by the glass carrier chip.

Although the above prior art can be used in related fields, the price is high or the precision is in lack. Hence, the prior arts do not fulfill users' requests on actual use.

### SUMMARY OF THE INVENTION

The main purpose of the present invention is to use a low-cost gene chip to precisely diagnose the cancer with an easy operated luminescence analysis and by weighting value for each gene according to its various role playing in the forming of the cancer.

To achieve the above purpose, the present invention is a complex cancer cells luminescence detection method, comprising steps of: (a) obtaining a gene chip of a nylon membrane chip, prepared through arranging oligonucleotide fragments of gene on a chip by using an arrayer and anchoring the oligonucleotide fragments of gene on the chip by using a rapid nucleotide anchoring device with a high power of 1200 joule; (b) obtaining a blood specimen of a patient, extracting ribonucleic acid from the blood specimen, reversely transcribing the ribonucleic acid into a complementary deoxyribonucleic acid (cDNA) and labeling the blood specimen as a signal matter; (c) after processing a hybridization reaction to the gene chip and the signal matter, processing a reaction test; (d) processing a chemical luminescence reaction to obtain a result image; and (e) processing a luminescence analysis to the result image and weighting a result of the luminescence analysis to precisely diagnose a cancer. Accordingly, a novel complex cancer cells luminescence detection method is obtained.

### BRIEF DESCRIPTION OF THE DRAWING

The present invention will be better understood from the following detailed description of the preferred embodiment according to the present invention, taken in con junction with the accompanying drawing, in which

FIG. 1 is the flow view showing the preferred embodiment according to the present invention.

### DESCRIPTION OF THE PREFERRED EMBODIMENT

The following description of the preferred embodiment is provided to understand the features and the structures of the present invention.

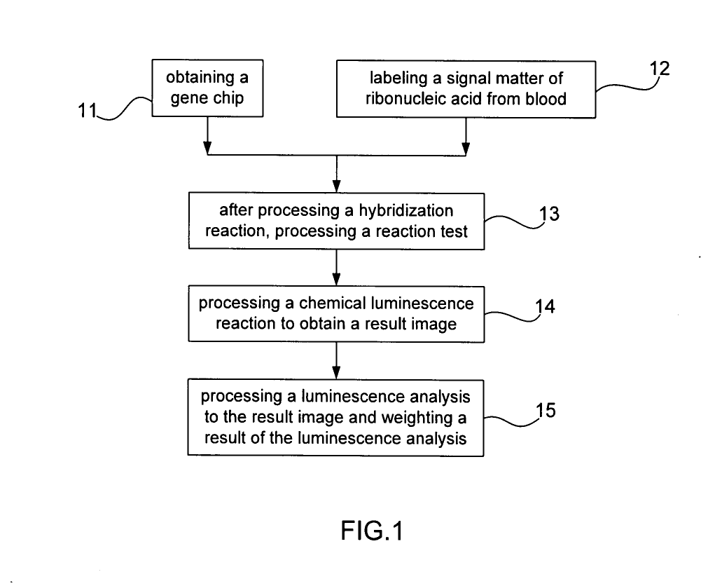

Please refer to FIG. 1, which is a flow view showing a preferred embodiment according to the present invention. As shown in the figure, the present invention is a complex cancer cells luminescence detection method, comprising the following steps:
(a) Preparing a gene chip 11: A gene chip is prepared. The gene chip is a nylon membrane chip, which is prepared through the following steps:
   (a1) Oligonucleotide fragments of gene are arranged on a chip by using an arrayer.
   (a2) And, the oligonucleotide fragments of gene are anchored on the chip by using a rapid nucleotide anchoring device with a high power of 1200 joule.
(b) Labeling a signal matter of ribonucleic acid from blood 12: The following steps are processed:
   (b1) A blood specimen of a patient is obtained;
   (b2) Ribonucleic acid is extracted from the blood specimen;
   (b3) The ribonucleic acid is reversely transcribed into cDNA;
   (b4) And, the blood specimen is labeled as a signal matter.
(c) Processing a hybridization reaction and a reaction test 13: The gene chip and the signal matter are processed with a hybridization reaction. After the hybridization reaction, a reaction test is processed.
(d) Processing a chemical luminescence reaction 14: After the reaction test, a chemical luminescence reaction is processed to obtain a result image.
(e) Processing a luminescence analysis and weighting 15: A luminescence analysis is processed to the result image. And the analysis result is weighted to accurately diagnose cancer.

Thus, a novel complex cancer cells luminescence detection method is obtained. The present invention has the following advantages:

(1) A nylon membrane chip is used to save cost.

(2) A labeling process and a hybridization reaction are simplified; and a sensitivity and a distinction of the gene chip are enhanced and are stable.

(3) A result image obtained through a chemical luminescence reaction can be automatically analyzed with a general luminescence analysis software, where a luminescence analysis has a lower cost and an easy operation.

(4) Each gene plays a various role in a cancer. Analysis result of the present invention is weighted according to various role each gene plays in the forming of the cancer. Thus, the cancer can be precisely diagnosed.

To sum up, the present invention is a complex cancer cells luminescence detection method, where a low-cost gene chip are used to diagnose the cancer precisely with an easy-operated luminescence analysis; and value for each gene is weighted according to its various role playing in the forming of the cancer.

The preferred embodiment herein disclosed is not intended to unnecessarily limit the scope of the invention. Therefore, simple modifications or variations belonging to the equivalent of the scope of the claims and the instructions disclosed herein for a patent are all within the scope of the present invention.

What is claimed is:

1. A complex cancer cells luminescence detection method, comprising steps of:
(a) obtaining a gene chip;
(b) obtaining a blood specimen, extracting ribonucleic acid from said blood specimen, reversely transcribing said ribonucleic acid into cDNA, and labeling said blood specimen as a signal matter;
c) after processing a hybridization reaction to said gene chip and said signal matter, processing a reaction test;
(d processing a chemical luminescence reaction to obtain a result image; and
(e) processing a luminescence analysis to said result image and weighting a result of said luminescence analysis.

2. The method according to claim 1 wherein said gene chip is a nylon membrane chip.

3. The method according to claim 1, wherein said gene chip is obtained through steps of:
(a1) arranging oligonucleotide fragments of gene on a chip by using an arrayer; and
(a2) anchoring said oligonucleotide fragments of gene on said chip by using a rapid nucleotide anchoring device.
